SEQUENCE LISTING (1 sequence)

SEQ ID NO: 1 - DNA, 34 bp, Artificial Sequence
  Synthetic construct
caactcggcaattagtaaagcctactgctttctc

(19) United States
(12) Patent Application Publication
(10) Pub. No.: US 2018/0051341 A1
(43) Pub. Date: Feb. 22, 2018

(54) Method for Reducing Sequencing Errors Caused by DNA Fragmentation

(71) Applicant: New England Biolabs, Inc., Ipswich, MA (US)

(72) Inventors: Thomas C. Evans, Jr., Topsfield, MA (US); Laurence Ettwiller, Beverly, MA (US); Lixin Chen, Beverly, MA (US); Pingfang Liu, Ipswich, MA (US)

(73) Assignee: New England Biolabs, Inc., Ipswich, MA (US)

(21) Appl. No.: 15/414,260

(22) Filed: Jan. 24, 2017

Related U.S. Application Data
(60) Provisional application No. 62/376,165, filed on Aug. 17, 2016.

(51) Int. Cl. C12Q 1/68

(52) U.S. Cl. CPC C12Q 1/6886; C12Q 1/6874; C12Q 1/6827; C12Q 1/6806; C12Q 2600/156; C12Y 301/21002; C12Y 207/07007; C12Y 605/01001; C12Y 302/02023

(57) ABSTRACT

Provided herein, among other things, is a method for reducing sequencing errors caused by mechanical DNA fragmentation. In some embodiments, this method may comprise: (a) obtaining a DNA sample; (b) mechanically fragmenting the DNA sample to produce a template; (c) treating the fragmented DNA with a plurality of DNA repair enzymes; and (d) obtaining a sequence of the fragmented DNA. The treatment step (c) reduces the number of fragmentation induced errors.

### CROSS-REFERENCING

This application claims the benefit of U.S. provisional application Ser. No. 62/376,165, filed on Aug. 17, 2016, which application is incorporated by reference herein in its entirety.

### BACKGROUND

Genomic variations in somatic cells can result in disease states including cancer. Thus, accurate tumor-associated variant detection, which may inform upon the potential to direct personalized treatments, is important for cancer diagnosis and prognosis. Next generation sequencing (NGS) has revolutionized variant identification and characterization. Nonetheless, owing to tumor heterogeneity and/or contamination by normal cells, somatic cancer variants are often found at low allelic frequencies confounding their identification.

Detection of low allelic frequency variants is achieved through deep sequencing and specialized data analysis algorithms that detect variants in a limited number of reads. Data analysis is challenged by artifactual errors that display the same low allelic frequency as cancer mutations with the level of artifactual errors defining the threshold for low allelic variant detection. Most sequencing errors are thought to result from PCR mistakes or sequencing miscalls. However, mutagenic DNA damage is recognized as a major source of sequencing errors only in samples that have already been fragmented and/or stored for a long time, e.g. FFPE (Do et al, Clin Chem. 2015 61, 64-71), ancient (Briggs et al., Nucleic Acids Research 2010 38, e87) and circulating tumor DNA (Newman et al., Nature Biotechnology 2016 34, 547-555). Samples that contain high quality, high molecular weight DNA were thought to be relatively free of damaged nucleotides and, as such, it was believed that sequencing errors due to DNA damage was not significant in such samples.

### SUMMARY

In general, it has been shown here that sequencing errors occur due to DNA damage that is the direct result of steps in sequencing protocols.

In some aspects, a method for reducing sequencing errors caused by mechanical DNA fragmentation is provided. In some embodiments this method may comprising: (a) obtaining a DNA sample; (b) mechanically fragmenting the DNA sample to produce a template; (c) treating the fragmented DNA with a plurality of DNA repair enzymes; and (d) obtaining a sequence of the fragmented DNA, wherein step (c) reduces the number of fragmentation induced errors.

In some embodiments, step (b) may be done by acoustic shearing.

In some embodiments, the treatment step (c) may be done by treating the template with enzymes that include at least one DNA glycosylase, an AP endonuclease, a DNA polymerase and a ligase.

In some embodiments, the enzymes may be in a mixture.

In some embodiments, the at least one DNA glycosylase may comprise oxoguanine glycosylase (OGG) or formamidopyrimidine-DNA glycosylase (FPG).

In some embodiments, step (d) may comprise amplifying the template.

In some embodiments, step (d) may comprise circularlizing the template.

In some embodiments, the method may comprise comprising analyzing sequence reads to identify a sequence variation.

In some embodiments, the DNA sample made from a clinical sample.

In some aspects a method for detecting damaged DNA in a sample is also provided. In some embodiments, this method may comprise: preparing a sequencing library from the damaged DNA;

sequencing the sequencing library to obtain a plurality of first Watson end sequence reads (R1s) and plurality of second complementary Crick sequence reads (R2s); for a selected locus, identifying those R1s and R2s that have a sequence abnormality; and detecting an imbalance between the number of R1s that have a sequence abnormality and the number of R2s that have a complementary sequence abnormality at the same position.

In some embodiments, the damaged DNA may be from a clinical sample, e.g., a formalin fixed paraffin embedded (FFPE) sample.

In some embodiments, the damaged DNA may be fragmented by acoustic shearing.

In some embodiments, the sequence abnormality may be a G to T transversion.

In some embodiments, the method may further comprise eliminating sequence reads or sequence abnormalities from further analysis if R1 and R2 reveal an imbalance.

### DESCRIPTION OF FIGURES

The patent or application file contains at least one drawing executed in color. Copies of this patent or patent application publication with color drawing(s) will be provided by the Office upon request and payment of the necessary fee.

Certain aspects of the following detailed description are best understood when read in conjunction with the accompanying drawings. It is emphasized that, according to common practice, the various features of the drawings are not to scale. On the contrary, the dimensions of the various features are arbitrarily expanded or reduced for clarity. Included in the drawings are the following figures:

FIG. 1A-1B. The Global Imbalance Value (GIV) score. (FIG. 1A) GIV score principle: Illumina adaptors are directional in nature, enabling consistent paired end sequencing within clusters. This property results in sequencing of the original strand orientation in the R1 reads (from the P5 adaptor) whereas the reverse complement orientation is read in the R2 reads (from the P7 adaptor). As damage affects only one base of a pair, damage such as 8-oxo-dG leads to an excess of G to T transversion errors when R1 is mapped to a reference genome, whereas, the R2 reads will show an excess of the reverse complement of G to T, i.e. C to A errors, instead. As a consequence, there is a global imbalance in the number of G to T variants in R1 compared to R2 sequences. This imbalance is specific to damage and is the basis of the GIV score (first panel). Contrasting with damage, true variations lead to no imbalance (second panel). (FIG. 2B) Variant profile: The fraction of G to T (upper panel) and C to A variants (lower panel) in R1 and R2 sequences were plotted as a function of the read (R1 or R2) and position (in bp). Acoustic shearing in different solutions generated various levels of G to T in R1 or C to A in R2. In all cases, treatment of the DNA sample with the repair enzyme cocktail reduced the number of G to T variants to baseline levels consistent with 8-oxo-dG damage being the cause of the excess G to T variants in the unrepaired samples.

FIG. 2A-2B. GIV scores (Y axis) for the 12 nucleotide substitution classes (X axis) for (FIG. 2A) The 1000 Genomes Project dataset and (FIG. 2B) A subset of the TCGA dataset. Each point represents the GIV score of a single sequencing run downsampled to 5 million reads. The solid gray line denotes a GIV of 1.5. The bimodal distribution of points observed in G to T and C to A substitution classes corresponds to sequencing runs with damage and without or limited amounts of damage, respectively.

FIG. 3A-3B. Target enrichment experiment. (FIG. 3A) G to T variant profiles across reads R1 and R2 with (red) and without (blue) DNA repair. (FIG. 3B) Number of variants per Mb of sequence per type (orange denotes G to T variants, blue denotes C to A variants, shades of gray denotes all the other variants) at frequencies indicated above the respective graph. (FIG. 3C) As in FIG. 3B, except that only R1 reads were used for variant calling.

FIG. 4A-4E. Variants identified in TCGA datasets. (FIG. 4A) ˜1800 tumor sequencing runs sorted by increasing GIV_{G}_{_}_{T} score. (FIG. 4B) Somatic variant profiles (Varscan) for the tumor samples sorted by increasing GIV_{G}_{_}_{T} scores. The fraction of G to T (orange) somatic variant calls is higher than C to A (blue) for most datasets and the fraction of G to T calls increases with increasing GIV_{G}_{_}_{T} score. (FIG. 4C) As in B using the high confidence somatic variant calls from Varscan. (FIG. 4D) As in FIG. 4B except the germline variant calls are represented. (FIG. 4E) Estimated false positive rate (in %, Y axis) of somatic G to T candidate variants found using Varscan as a function of the GIV_{G}_{_}_{T} score (X axis).

FIG. 5A-5E. Shearing conditions affect the degree of damage. (FIG. 5A) Overall fraction of G to T variants (normalized to the total number of G) for R1 and the reverse complement of R2 sequences. Different buffers were used during acoustic shearing (x-axis). Data in orange were from samples that were not repaired and data in blue were from samples that were repaired. Each point corresponded to a random sampling of 2 million sequence positions. All samples were derived from the same human genomic DNA. (FIGS. 5B, 5C and 5D) Variant profiles and context specificity across R1 and R2 sequences for G to T (FIG. 5B), A to T (FIG. 5C) and C to T (FIG. 5D) for samples sheared in water (blue), 0.1×TE (orange) and 1×TE (gray). G to T transversions were elevated in R1 sequences in all contexts. In all three variant cases, an elevated fraction (red arrow) was found at the 5′ end of R2 sequences in a 5′ T context. (FIG. 5E) Correlation between the degree of damage and GIV for G to T variant (GIV_{G}_{_}_{T}) and T to A variant (GIV_{T}_{_}_{A}).

FIG. 6A-6B. (FIG. 6A) Frequency of G to T transversions in samples sheared in water (gray), 1 mM Tris (orange), 1 mM Tris+0.1 mM EDTA (light blue), 10 mM Tris (green), 10 mM Tris+0.1 mM EDTA (yellow) and 10 mM Tris+1 mM EDTA (dark blue). The samples sheared in water were also treated with repair enzymes to identify the baseline G to T transversions from undamaged DNA (red). (FIG. 6B) G to T variant frequency as a function of read position for R1 sequences and R2 sequences in samples sheared in water (gray), 1 mM Tris (orange), 1 mM Tris+0.1 mM EDTA (light blue), 10 mM Tris (green), 10 mM Tris+0.1 mM EDTA (yellow) and 10 mM Tris+1 mM EDTA (dark blue). The samples sheared in water were also treated with the repair enzyme cocktail to identify the baseline G to T transversion from undamaged DNA (red). Imbalances between R1 and R2 sequences were identified in all conditions with the exception of the repaired sample (red). The level of imbalance is inversely correlated with buffer concentration demonstrating that the buffering capacity of the solution during acoustic shearing was the major modulator of damage.

FIG. 7A-7B. (FIG. 7A) Correlation between Phred quality cutoff and GIV_{G}_{_}_{T} (blue) and GIV_{C}_{_}_{A} (black) for genomic samples sheared in water or various buffer conditions (0.1×TE, 1×TE) and repaired sample sheared in 1×TE. The fraction of errors due to damage increased with the confidence of base calling, explaining the increase in GIV_{G}_{_}_{T} with increasing phred quality cutoff in untreated samples. This result indicates that errors induced by damage cannot simply be removed by elevating the stringency of base calling. (FIG. 7B) Effect of sequencing depth (number of reads) on GIV scores. Sequencing reads from samples sonicated in water (red), 0.1×TE buffer (blue) and 1×TE buffer (orange) range from 0.1 million reads to 6 million reads. For each down-sampled experiment, GIV scores were calculated and plotted. The dotted line (at 2 million reads) indicates stabilization of the GIV score and the lower limit of reads required to accurately calculate a GIV score.

FIG. 8A-8B. Damage in the TCGA dataset. (FIG. 8A) G to T variant frequency as a function of read position in R1 and R2 for sequence context ApG (panel 1), TpG (panel 2) and GpG (panel 3). (FIG. 8B) Same as in A except for the CpG context. (FIG. 8C) Same as in A except for the GpA (panel 1), GpC (panel 2), GpT (panel 3) and GpG (panel 4) context. Each line corresponds to one TCGA sample. The imbalance of G to T on the R1 compared to R2 sequencing reads can be observed in all contexts for most of the samples. We observed an elevated G to T on the 5′ end of the R2 sequence reads corresponding to an elevated C to A on the 3′ end of the original fragments. Contrary to G to T variants of R1 sequences, this elevated C to A frequency has a strong sequence context at the TpG context (red arrow) and position specificity within the first 20 bp of the R2 reads.

FIG. 9. Damage in the TCGA dataset. A to T variant frequency as a function of read position in the R1 and R2 reads for sequence context ApA (panel 1), ApC (panel 2) and ApT (panel 3) and ApG (panel 4). Red arrows denote elevated variant frequency at the 5′ end of R2. The elevated A to T frequency 5′ of the R2 reads correspond to damage leading to a T to A transversion at the 3′ end of the original fragments.

FIG. 10A-10C. Damage in the most recently submitted TCGA runs. (FIG. 10A) GIV scores for all the 12 nucleotide substitution classes. The dotted line denotes a GIV score of 1 which corresponds to no damage. The solid line denotes a GIV score of 1.5 corresponding to a limit of significant damage. (FIG. 10B) G to T variation frequency as a function of the position on the read (bp) in R1 and R2 sequences. (FIG. 10C) A to T variation frequency as a function of the position on the read (bp) in R1 and R2 sequences. The elevated A to T frequency 5′ of the R2 reads correspond to damage leading to a T to A transversion at the 3′ end of the original fragments.

FIG. 11A-11E. (FIG. 11A) Distribution of the GIV_{C}_{_}_{T} scores in the ˜1800 tumor samples from the TCGA dataset. (FIG. 11B) Profile of somatic variant calls for the ˜1800 tumor samples analyzed and organized by increasing GIV_{C}_{_}_{T} scores. The fraction of somatic variant calls of C to T types (orange) is higher when compared to G to A (light blue) for a subset of the datasets and the overall fraction of somatic variant calls of C to T types increase with the increase of GIV_{G}_{_}_{T} score. (FIG. 11C) Same as in B except using the profile of high confidence somatic variant calls instead of somatic variant calls. (FIG. 11D) Same as in B, except using the profile of germline variant calls instead of somatic variant calls. (FIG. 11E) Estimated false positive rate of somatic variant calls of the C to T type (in percent) as a function of the GIV_{C}_{_}_{T}.

FIG. 12 shows the distribution in the percentage of high confident candidate variants of type G to T or C to A for weakly or no damaged datasets (GIV_{G}_{_}_{T}<1.5, blue) compared to heavily damaged datasets (GIV_{G}_{_}_{T}>4.5, red). Significant difference in distribution was assessed using KS test with ns (pᵥₐₗᵤₑ>0.05) *(pᵥₐₗᵤₑ between 0.01 and 0.05) and **(pᵥₐₗᵤₑ between 0.01 and 0.001).

### DESCRIPTION

Before describing exemplary embodiments in greater detail, the following terms are described so as to illustrate the meaning and scope of their use in the description.

Numeric ranges are inclusive of the numbers defining the range. Unless otherwise indicated, nucleic acids are written left to right in 5′ to 3′ orientation; amino acid sequences are written left to right in amino to carboxy orientation, respectively.

Unless defined otherwise, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Markham, THE HARPER COLLINS DICTIONARY OF BIOLOGY, Harper Perennial, N.Y. (1991) provide one of skill with the general meaning of many of the terms used herein. Still, certain terms are defined below for the sake of clarity and ease of reference.

It must be noted that as used herein and in the appended claims, the singular forms “a”, “an”, and “the” include plural referents unless the context clearly dictates otherwise. For example, the term “a primer” refers to one or more primers, i.e., a single primer and multiple primers. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

The term “DNA sample,” as used herein denotes a sample containing DNA. A DNA sample used herein may be complex in that they contain multiple different molecules that contain sequences. Genomic DNA, RNA (and cDNA made from the same) from a mammal (e.g., mouse or human) are types of complex samples. Complex samples may have more then 10⁴, 10⁵, 10⁶ or 10⁷ different nucleic acid molecules. A DNA sample may originate from any source such as genomic DNA or cDNA. Any sample containing nucleic acid, e.g., genomic DNA made from tissue culture cells or a sample of tissue, may be employed herein. Any sample containing nucleic acid, e.g., genomic DNA made from tissue culture cells, a sample of tissue, a clinical, environmental, or other type of sample may be employed herein.

The term “mixture”, as used herein, refers to a combination of elements, that are interspersed and not in any particular order. A mixture is heterogeneous and not spatially separable into its different constituents. Examples of mixtures of elements include a number of different elements that are dissolved in the same aqueous solution and a number of different elements attached to a solid support at random positions (i.e., in no particular order). A mixture is not addressable. To illustrate by example, an array of spatially separated surface-bound polynucleotides, as is commonly known in the art, is not a mixture of surface-bound polynucleotides because the species of surface-bound polynucleotides are spatially distinct and the array is addressable.

The term “amplifying” as used herein refers to the process of synthesizing nucleic acid molecules that are complementary to one or both strands of a template nucleic acid. Amplifying a nucleic acid molecule may include denaturing the template nucleic acid, annealing primers to the template nucleic acid at a temperature that is below the melting temperatures of the primers, and enzymatically elongating from the primers to generate an amplification product. The denaturing, annealing and elongating steps each can be performed one or more times. In certain cases, the denaturing, annealing and elongating steps are performed multiple times such that the amount of amplification product is increasing, often times exponentially, although exponential amplification is not required by the present methods. Amplification typically requires the presence of deoxyribonucleoside triphosphates, a DNA polymerase enzyme and an appropriate buffer and/or co-factors for optimal activity of the polymerase enzyme. The term “amplification product” refers to the nucleic acid sequences, which are produced from the amplifying process as defined herein.

The terms “determining”, “measuring”, “evaluating”, “assessing,” “assaying,” and “analyzing” are used interchangeably herein to refer to any form of measurement, and include determining if an element is present or not. These terms include both quantitative and/or qualitative determinations. Assessing may be relative or absolute. “Assessing the presence of” includes determining the amount of something present, as well as determining whether it is present or absent.

A “plurality” contains at least 2 members. In certain cases, a plurality may have at least 2, at least 5, at least 10, at least 100, at least 100, at least 10,000, at least 100,000, at least 10⁶, at least 10⁷, at least 10⁸ or at least 10⁹ or more members.

The term “strand” as used herein refers to a nucleic acid made up of nucleotides covalently linked together by covalent bonds, e.g., phosphodiester bonds. In a cell, DNA usually exists in a double-stranded form, and as such, has two complementary strands of nucleic acid referred to herein as the “top” and “bottom” strands. In certain cases, complementary strands of a chromosomal region may be referred to as “plus” and “minus” strands, the “first” and “second” strands, the “coding” and “noncoding” strands, the “Watson” and “Crick” strands or the “sense” and “antisense” strands. The assignment of a strand as being a top or bottom strand is arbitrary and does not imply any particular orientation, function or structure. The nucleotide sequences of the first strand of several exemplary mammalian chromosomal regions (e.g., BACs, assemblies, chromosomes, etc.) is known, and may be found in NCBI's Genbank database, for example.

The term “sequencing”, as used herein, refers to a method by which the identity of at least 10 consecutive nucleotides (e.g., the identity of at least 20, at least 50, at least 100 or at least 200 or more consecutive nucleotides) of a polynucleotide are obtained.

The term “next-generation sequencing” refers to the so-called parallelized sequencing-by-synthesis or sequencing-by-ligation platforms currently employed by Illumina, Life Technologies, Pacific Biosciences and Roche etc. Next-generation sequencing methods may also include Nanopore sequencing methods or electronic-detection based methods such as Ion Torrent technology commercialized by Life Technologies.

The term “potential sequence variant” or “sequence abnormality” refers to a potential variation in a DNA sequence, as determined by examining sequence reads. All sequencing methods inherently produce errors that are generated by, e.g., damage caused by DNA fragmentation. These problems limit one's ability to identify true or real sequence variation or sequence abnormalities in the sample and, indeed, because of these problems many of the current methods are reported to have a low specificity or a high false positive rate. Against this background, it can be difficult to detect real genetic variants, particularly low frequency variants, with high specificity and sensitivity. In some cases, a variant is present at a frequency of less than 10%, relative to other molecules in the sample. In some cases, a variant may be a first allele of a polymorphic target sequence, where, in a sample, the ratio of molecules that contain the first allele of the polymorphic target sequence compared to molecules that contain other alleles of the polymorphic target sequence is 1:100 or less, 1:1,000 or less, 1:10,000 or less, 1:100,000 or less or 1:1,000,000 or less. A variant can be a substitution, insertion, deletion, inversion, transversion, transition, duplication, conversion, translocation or complex event where more than one of these processes has occurred.

The terms “aberrant nucleotide” and “damaged nucleotide” refer to derivatives of adenine (A), cytosine (C), guanine (G), and thymine (T) in which the base of the nucleotide has been altered in a way that allows it to pair with a different base. In normal base pairing, A base pairs with T and C base pairs with G. Bases may be altered by oxidation, alkylation or deamination in a way that effects base pairing. 7,8-dihydro-8-oxoguanine (8-oxo-dG) is a derivative of guanine that base pairs with adenine instead of cytosine and thus generates GC to TA transversion after replication. Uracil is formed by deamination of cytosine and can base pair with adenine, leading to a C to T change after replication. Other examples or damaged nucleotide that are capable of mismatched pairing include, but are not limited to, 2,6-diamino-4-hydroxy-5-formamidopyrimidine, 3-methyladenine, 7-methylguanosine, hypoxanthine (formed from deamination of adenine), xanthine (formed by deamination of guanine), 8-oxoadenine (O8A) and O(6)-methylguanine. Mismatched pairs caused by damaged bases include AχA, GχG, AχG, CχC, TχT, CχT, AχC and GχT base pairs.

The term “DNA repair enzymes” includes enzymes that are capable of recognizing and repairing damaged nucleotides. In some cases, such an enzyme may reverse the damage in the base directly (e.g., O(6)-methylguanine-DNA methyltransferase (MGMT), which demethylates O(6)-methylguanine), by base excision repair (which involves removing the damaged base with a DNA glycosylase to produce an abasic site, cleaving the site using an AP endonuclease, replacing the damaged nucleotide using a polymerase and sealing the site using a ligase) or by mismatch repair. 3-methyladenine DNA glycosylase I (TAG), uracil DNA glycosylase (UDG), thymine-DNA glycosylase (TDG), oxoguanine glycosylase (OGG) or formamidopyrimidine-DNA glycosylase (FPG) are examples of glycosylases (see, e.g., Brooks et al Biochim Biophys Acta. 2013 January; 1834(1): 247-271 and Jacobs et al Chromosoma. 2012 121:1-20) that can be employed in conjunction with a ligase, polymerase and AP endonuclease to repair damaged bases. In some embodiments, a DNA repair enzyme may recognize a purine-purine mismatch (AχA, GχG or AχG), a pyrimidine-pyrimidine mismatch (CχC, TχT or CχT) or a purine-pyrimidine mismatch (AχC and GχT) (see also U.S. Pat. No. 7,700,283, U.S. Pat. No. 8,158,388 and US 2010/0173364). One example of a DNA repair kit contains Taq DNA ligase, E. coli endonuclease IV, Bst DNA polymerase, E. coli Fpg, E. coli UDG, T4 PDG, and E. coli Endonuclease VIIITaq DNA ligase, E. coli endonuclease IV, Bst DNA polymerase, E. coli Fpg, E. coli UDG, T4 PDG, and E. coli Endonuclease VIII. DNA repair kits are available commercially (see for example PreCR® New England Biolabs, Ipswich, Mass.).

Commonly used mechanical fragmentation methods, such as acoustic shearing, damage DNA. While acoustic shearing is commonly used (Covaris shearing), other forms of mechanical shearing or enzymatic shearing or chemical shearing may also be used. These too may be capable of damaging DNA.

In many samples the frequency of damaged nucleotides caused by fragmentation of DNA is similar to the frequency of “true” mutations, e.g., mutations that are associated with cancer and, as such, it can be difficult to determine whether a potential sequence variation is a true mutation or due to DNA fragmentation. Fragmentation damage is shown here to be a pervasive cause of sequencing errors. The examples show that treatment of a fragmented sample with a DNA glycosylase such as oxoguanine glycosylase (OGG) or formamidopyrimidine-DNA glycosylase (FPG) prior to amplification can virtually eliminate the damage caused by fragmentation such as mechanical fragmentation, thereby allowing one to identify true sequence variations with greater confidence.

Sequence variations generated during the preparation of sequencing libraries can be identified and potentially eliminated if there is an imbalance between the read 1 sequence reads that have the sequence variation and the read 2 sequence reads that have the complementary sequence variation.

Other compositions, systems, methods, features and advantages may become apparent to one with skill in the art upon examination of the following figures and detailed description. It is intended that all such additional systems, methods, features and advantages be included within this description and be within the scope of the invention.

Provided herein, among other things, is a method for reducing sequencing errors caused by DNA fragmentation. In some embodiments, the method may comprise mechanically fragmenting a DNA sample to produce a template, treating a template with one or more DNA repair enzymes to repair a damaged nucleotide that is capable of mismatched pairing optionally, amplifying the template, and sequencing the amplified template to produce a plurality of sequence reads. Treatment of the template in step (c) reduces the number of sequencing errors in the sequence reads.

The DNA in the sample (prior to fragmentation) may be relatively intact and, in some embodiments, may have a median length of at least 5 kb or at least 10 kb. The template molecules may have a median length in the range of 100 bp to 1.5 kb bp, e.g., 100 bp to 800 bp. The mechanical fragmentation may be done by any suitable method, e.g., by hydrodynamic shearing, acoustic shearing (see, e.g., WO2016/061098), or sonication. In some embodiments, the DNA may be fragmented to a size in the range of 5 kb to 30 kb. These fragments may be circularized prior to sequencing in some embodiments (e.g., PacBio SMRT sequencing (Pacific Biosystems, Menlo Park, Calif.).

In some embodiments the plurality of DNA repair enzymes may comprise a mismatch-specific DNA glycosylase and, in some embodiments, the template may be repaired by treating the template with enzymes that include a DNA glycosylase, an AP endonuclease, a DNA polymerase and a ligase. These enzymes may be in a mixture.

In some embodiments, the template may be treated with oxoguanine glycosylase (OGG) or formamidopyrimidine-DNA glycosylase (FPG) (as reviewed in Faucher et al Int. J. Mol. Sci. 2012 13: 6711-6729). The template may also be treated with a thymine-DNA glycosylase and/or uracil-DNA glycosylase.

The DNA may be obtained from any suitable sample and in some embodiments may be extracted from a clinical sample such as a tumor biopsy, FFPE sample or blood, for example. In some cases, the DNA can be extracted from a culture of cells, e.g., a cell line. In other cases, the cells may be isolated from an individual (e.g., a patient or the like). The cells may be isolated from a soft tissue or from a bodily fluid, or from a cell culture that is grown in vitro. In particular embodiments, the DNA may be isolated from a soft tissue such as brain, adrenal gland, skin, lung, spleen, kidney, liver, spleen, lymph node, bone marrow, bladder, stomach, small intestine, large intestine or muscle, etc. Bodily fluids include blood, plasma, saliva, mucous, phlegm, cerebral spinal fluid, pleural fluid, tears, lactal duct fluid, lymph, sputum, cerebrospinal fluid, synovial fluid, urine, amniotic fluid, and semen, etc.

In some embodiments, the template can be amplified by PCR. In some embodiments, the method may comprise ligating adaptors onto the template after it has been treated with the one or more DNA repair enzymes. In these embodiments the amplification step may comprises amplifying the template using primers that hybridize to the adaptors or their complement to produce the amplified template. As would be apparent, the adaptors ligated to the fragments and/or the primers used for amplification may be compatible with use in a next generation sequencing platform, e.g., Illumina's reversible terminator method, Roche's pyrosequencing method (454®), Life Technologies' sequencing by ligation (the SOLiD® platform), Life Technologies' Ion Torrent® platform or Oxford Nanopore's MinIon® system. Examples of such methods are described in the following references: Margulies et al (Nature 2005 437: 376-80); Ronaghi et al (Analytical Biochemistry 1996 242: 84-9); Shendure (Science 2005 309: 1728); Imelfort et al (Brief Bioinform. 2009 10:609-18); Fox et al (Methods Mol Biol. 2009; 553:79-108); Appleby et al (Methods Mol Biol. 2009; 513:19-39) and Morozova (Genomics. 2008 92:255-64), which are incorporated by reference for the general descriptions of the methods and the particular steps of the methods, including all starting products, reagents, and final products for each of the steps. The present method may be used on any sequencing platform, including those that are based on sequencing-by-synthesis (i.e., by extending a primer that is hybridized to a template). Some sequencing methods sequence a circularized template and, as such, the method may comprise circularizing the fragments prior to sequencing (before or after adaptors have been ligated onto the ends of the fragments).

In some embodiments, the sequencing is done to a depth of at least 10× or at least 20×. The sequencing may be paired-end sequencing.

The plurality of sequence reads may be analyzed to identify a sequence variation. The workflow for this step may involve trimming sequences, aligning the reads to a reference sequence and then interrogating the aligned data at each position of interest (see, e.g., Sutton et al Laboratory Investigation 2014 94, 1173-1183), although a variety of different methods can be implemented (see, generally, Altmann et al Bioinformatics. 2011 27: 77-84). By incorporating the repair step into the method, it is believed that true sequence variants can be called with a greater confidence than without the repair step.

Also provided is a method for eliminating a sequence artefact, i.e., determining that a sequence variation is not a “true” sequence variation in the sample, but rather a sequence artifact caused by DNA damage. In some embodiments, this method may comprise preparing a sequencing library from the damaged DNA; sequencing the sequencing library to obtain a plurality of first Watson end sequence reads (R1s) and plurality of second complementary Crick sequence reads (R2s); for a selected locus, identifying those R1s and R2s that have a sequence abnormality; and detecting an imbalance between the number of R1s that have a sequence abnormality and the number of R2s that have a complementary sequence abnormality at the same position. In these embodiments, the method may comprise preparing a sequencing library from a DNA sample, performing paired end sequencing on the sequencing library to obtain a plurality of first end sequence reads and a plurality of second end sequence reads, identifying a potential sequence variation in the sample; and eliminating the potential sequence variation if there is an imbalance between the first end sequence reads that have the sequence variation and the second end sequence reads that have the complementary sequence variation. This method may comprise calculating the ratio of first end sequence reads (or strands) that have the sequence variation relative to the number of second end sequence reads (or strands) that a complementary sequence variation (i.e., a complementary variation at the corresponding position). If the ratio is above a threshold (e.g., above 1.5, above 2.0 or above 3.0), then the potential sequence variation is likely due to DNA damage during sample preparation and, as such, can be eliminated.

Also provided is a method for quantifying global DNA damage in a sample. In some embodiments, this method may comprise sequencing a library as discussed above, e.g., by paired end sequencing, determining the total number of variants of a first type (e.g., G to T substitutions) in the first end sequence reads and the total number of the variants of the complementary type (e.g., C to A substitutions) in the second end sequence reads; and calculating a ratio of the total number of variants of a first type in the first end sequence reads and the total number of the variants of the complementary type in the second end sequence reads. The ratio indicates the amount of damage in the sample. If the ratio is above a threshold (e.g., above 1.5, above 2.0 or above 3.0), then the sample may be too damaged to provide reliable results.

Also provided is a method for detecting damaged DNA in a sample. In some embodiments, this method may comprise preparing a sequencing library from the damaged DNA, sequencing the library to obtain a plurality of first end sequence reads and a plurality of second end sequence reads; for a selected locus, identifying the first end sequence reads that have a sequence variation and the second end sequence reads that have the sequence variation and detecting an imbalance between the number of first end sequence reads that have a sequence variation to the number of second end sequence reads that have a complementary sequence variation at the same position. If there is an imbalance then the variation may be due to DNA damage. In these embodiments, the method comprises providing the ratio of the number of first end sequence reads (or strands) that have a sequence variation to the number of second end sequence reads (or strands) that have a complementary sequence variation, thereby quantifying the amount of damaged DNA in the sample. In some embodiments, the method may comprise performing the method using a second sequence library made using a different method, and comparing the ratios obtained for both methods, thereby quantitatively determining if the test method has increased or reduced the number of said damaged nucleotides in the sample.

In some embodiments, the method may be one for determining whether a DNA sequence from a sample genome has a somatic mutation or an error due to sample storage, sample sequencing or sample library preparation. In these embodiments, the method may comprise: (i) performing paired-end sequencing of a sample genome; (ii) comparing read 1 and read 2; (iii) identifying differences between read 1 and read 2 sequences of the sample genome; (iv) quantifying the imbalance between the nucleotide differences of read 1 and read 2 of the sample genome; (v) determining whether the imbalance observed in (iv) is above a particular threshold. A method to improve the accuracy of sequence determination of a sample template damaged by sample storage, sample sequencing or sample library comprising treatment of the sample template to remove a modified nucleotide base, which if not removed, would result in the synthesis of a complementary DNA strand that will differ from the complementary DNA strand that would have been synthesized opposite a non-modified nucleotide is also provided.

All references cited herein are incorporated by reference.

### EXAMPLES

The following examples are given for the purpose of illustrating various embodiments of the invention and are not meant to limit the invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

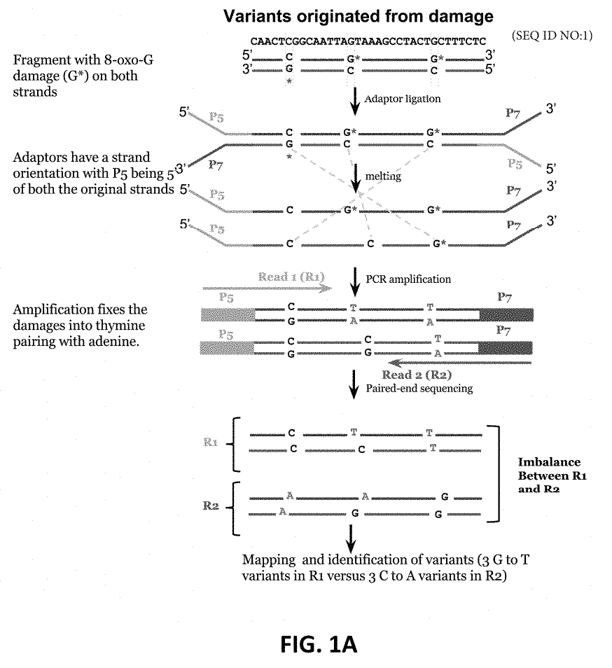

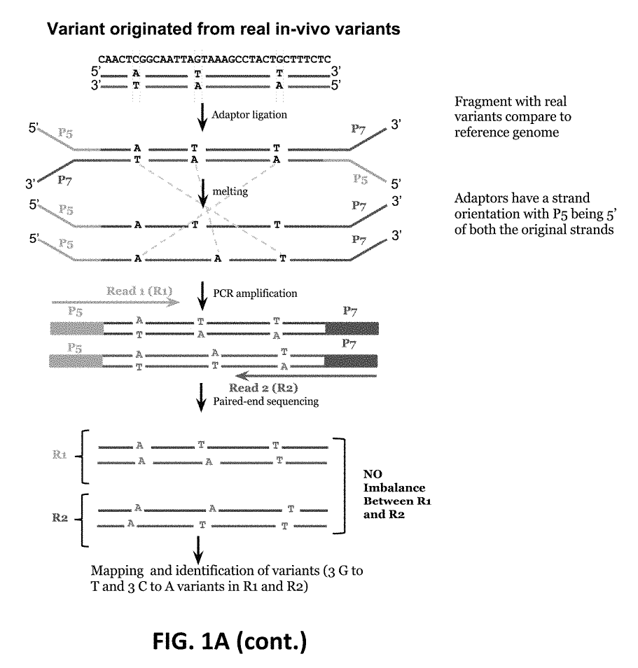

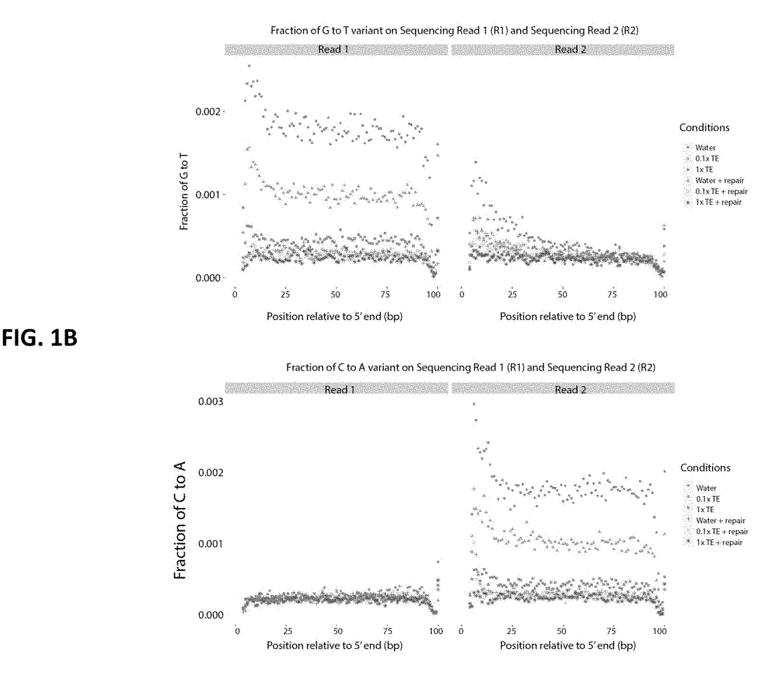

DNA damage in a number of different samples was measured. These measurements were made on the principle that mutagenic damage leads to a global imbalance between variants detected in read 1 (R1) and read 2 (R2) in paired-end sequencing (FIG. 1A) was used. The degree of this imbalance directly correlates with the amount of damage present in a sample. An analysis strategy based on this imbalance was devised to deconvolute both the origin and orientation of variants and computed a metric, the Global Imbalance Value (GIV) score, which is indicative of damage.

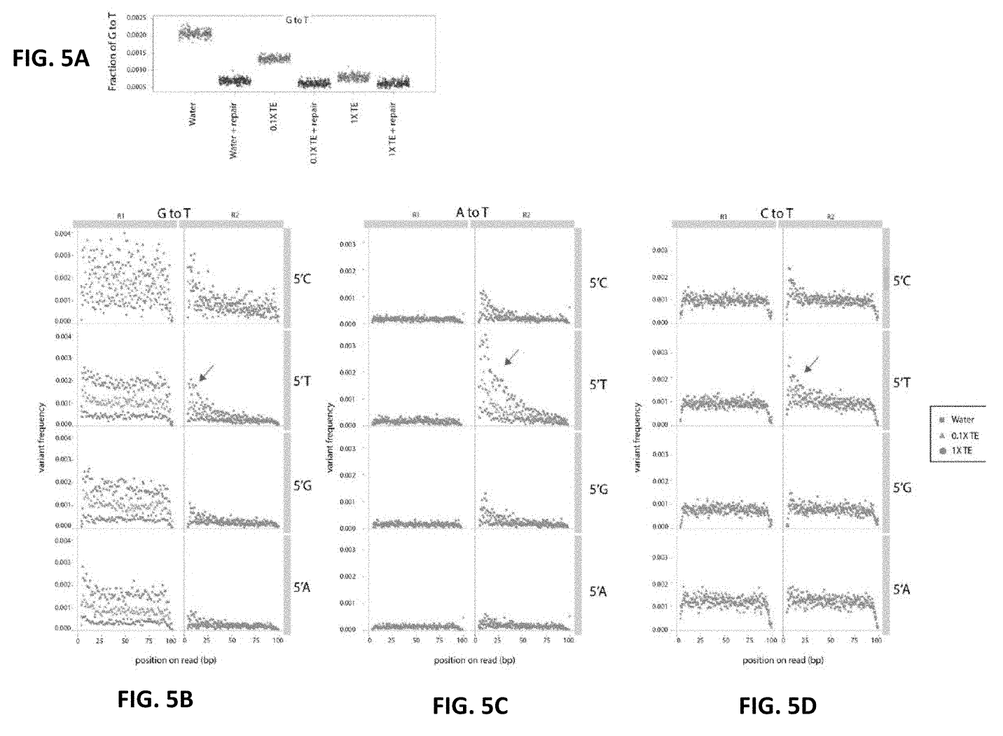

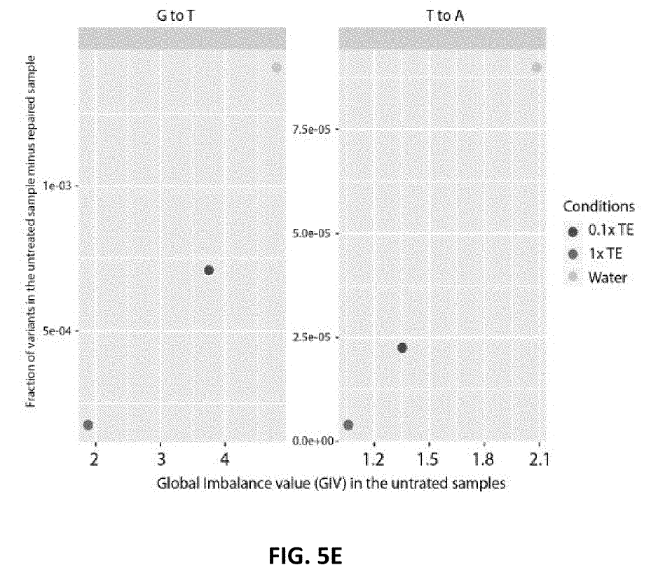

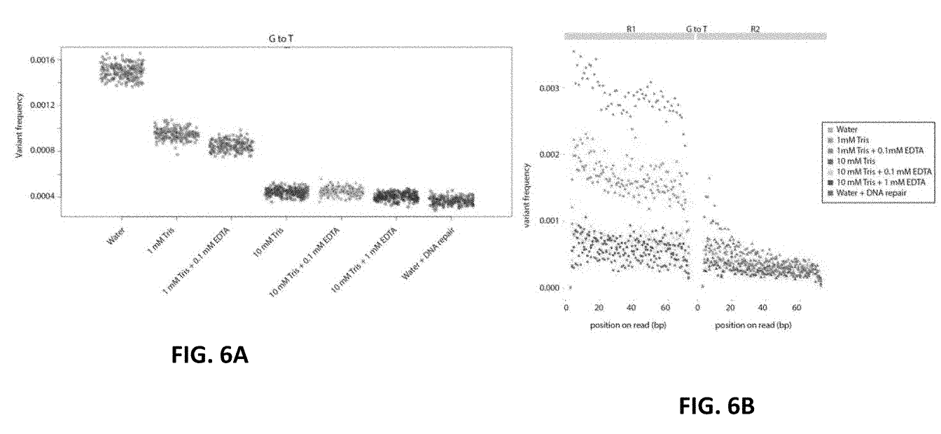

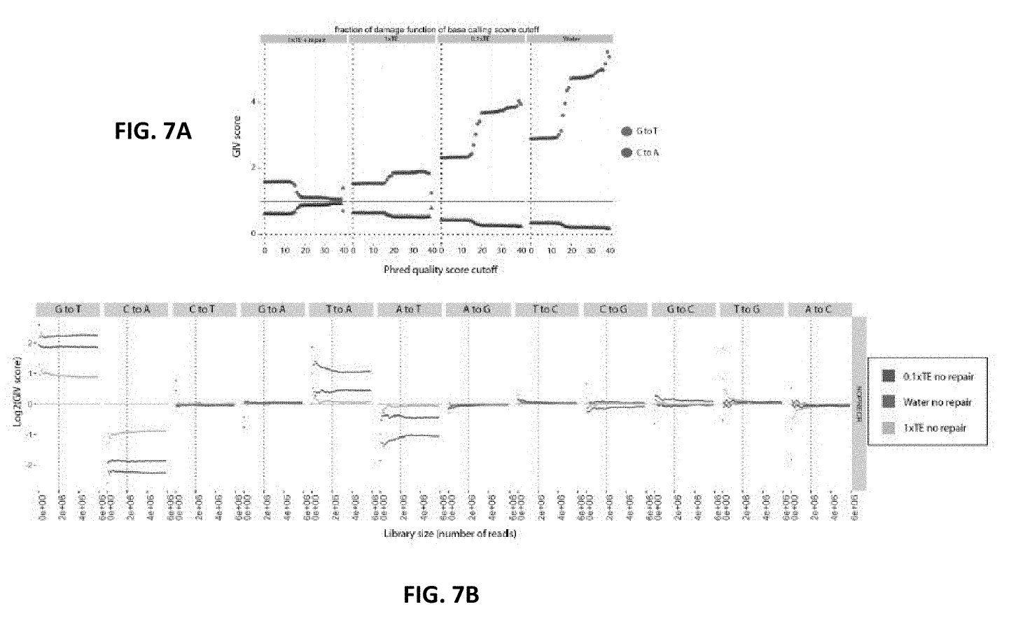

The algorithm produces 12 GIV scores, one per variant type. In this study, a GIV score above 1.5 is defined as damaged. At this GIV score, there are 1.5 times more variants on R1 than R2, suggesting that at least one third of the variants are erroneous. Undamaged DNA samples have a GIV score of 1. To experimentally validate the GIV score and provide an independent damage quantification, we used human genomic DNA containing various amounts of 7,8-dihydro-8-oxoguanine (8-oxo-dG) resulting in G to T transversions after amplification. The damaged DNA was also treated with an enzyme cocktail that repairs DNA damage prior to library preparation. Sequencing the same sample with and without DNA repair enzyme treatment quantified the rate of erroneous variants specifically introduced by damage. The G to T transversion frequency varied according to library preparation conditions (FIGS. 5A-5E and 6A-6B). Importantly, excess G to T variants were only observed in R1 sequences while C to A variants were in excess in R2 sequences leading to a GIV_{G}_{_}_{T} score>1 (FIG. 1B). Repair enzyme treatment abolished this imbalance and reduced the GIV_{G}_{_}_{T} score to 1. Importantly, the GIV score correlated with the variant excess measured experimentally (FIG. 5E), demonstrating that the GIV score can be used to accurately estimate the extent of damage in publically available datasets. It was estimated that the GIV score calculation is accurate at >2 million reads (FIG. 7B).

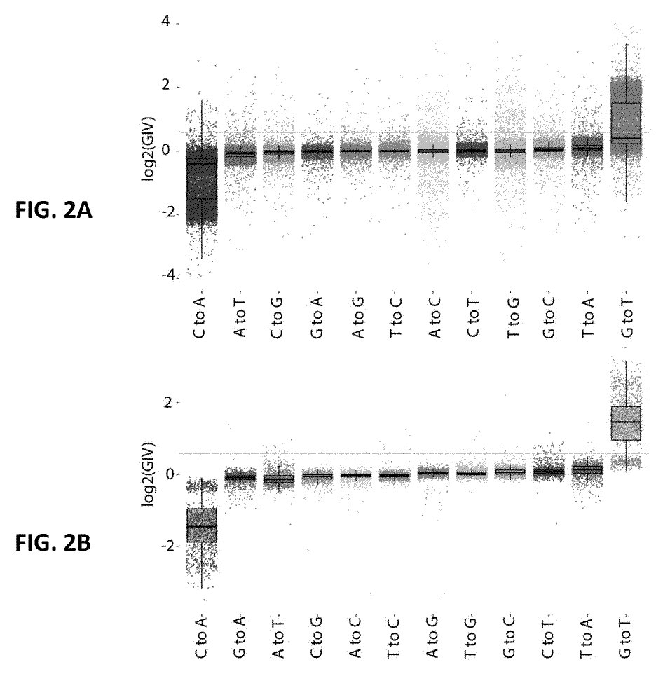

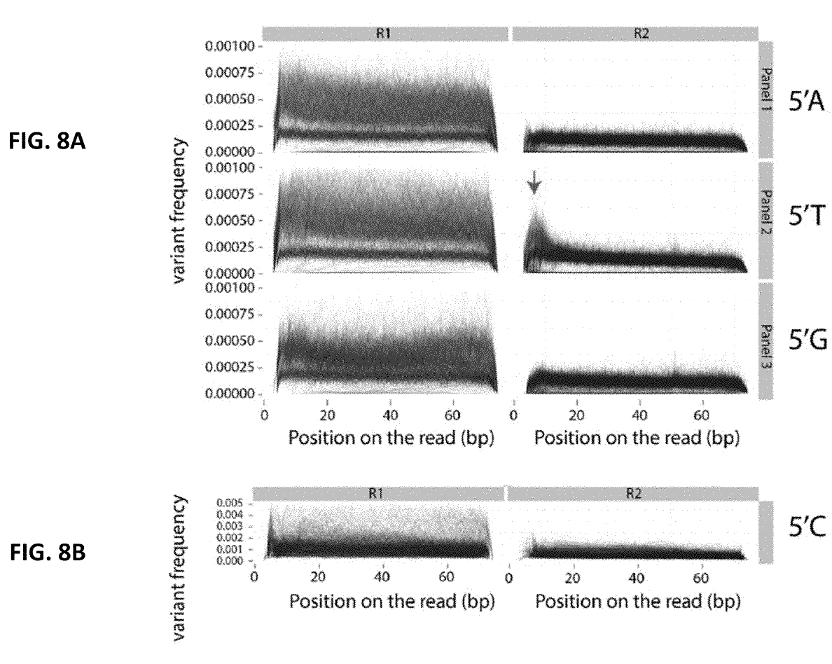

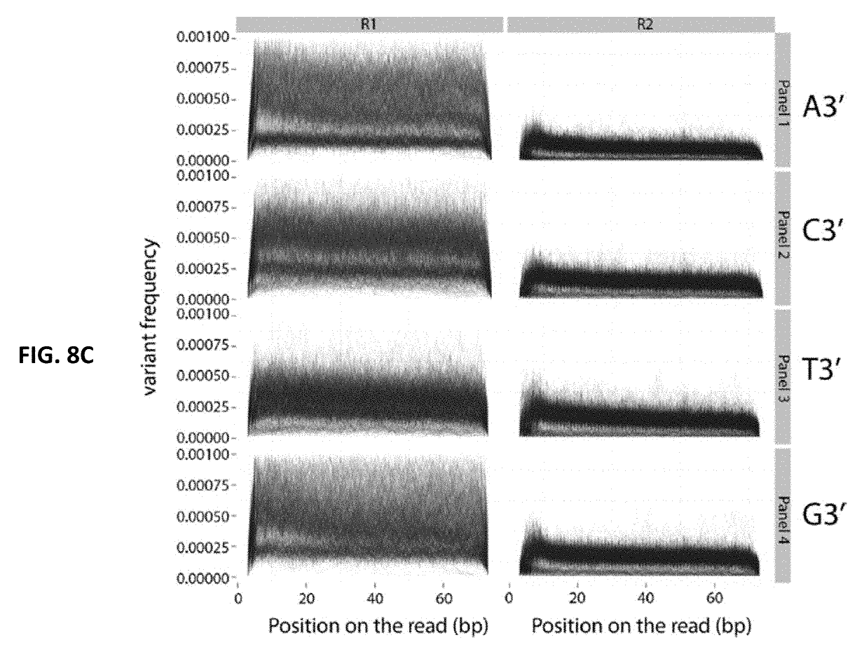

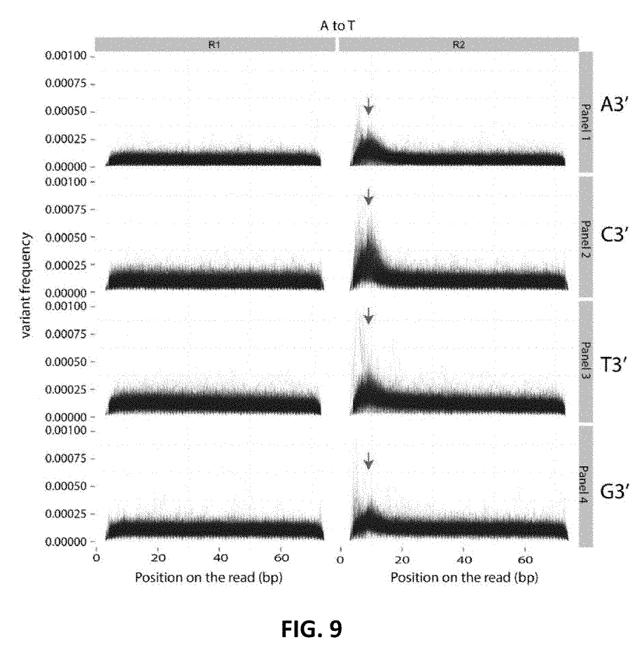

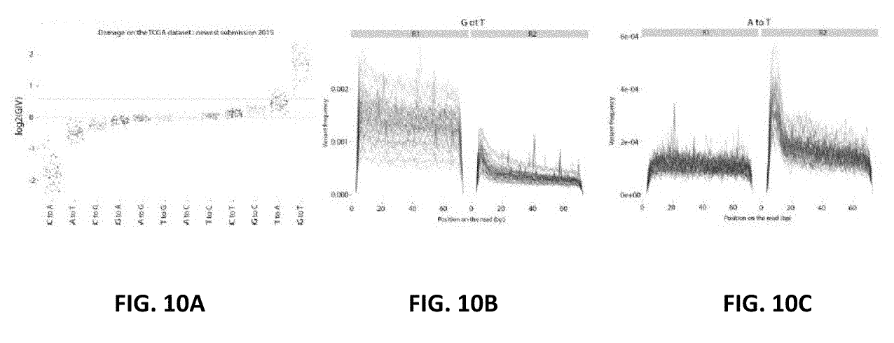

To estimate the extent of damage in public datasets, the GIV scores of individual sequencing runs from the 1000 Genomes Project and a subset of the TCGA dataset were determined. Both datasets showed widespread damage, particularly those leading to an excess of G to T variants (FIG. 2A-2B). Specifically, 41% of the 1000 Genomes Project datasets had a GIV_{G}_{_}_{T} score≧1.5 indicative of damaged samples (FIG. 2A). Furthermore, 73% of the TCGA sequencing runs showed extensive damage with a GIV_{G}_{_}_{T}>2. This indicates that the majority of G to T observations are erroneous and establishes damage as a pervasive cause of errors in these datasets (FIG. 2B). Further, we found no nucleotide context specificity of G to T imbalances in these datasets (FIG. 8A-8C). Additionally, an A to T imbalance (FIG. 9) leading to GIV_{T}_{_}_{A}>1.5 and a C to T imbalance (GIV_{C}_{_}_{T}>1.5) were detected in 0.5% and 3% of the TCGA dataset, respectively. Finally, recent submissions to TCGA (November-December 2015) displayed similar G to T imbalances and accentuated A to T imbalances (FIG. 10A-10C). These results confirm that most publicly available datasets, including recent submissions to TCGA, have signatures of damage leading to erroneous calls in at least one third of the G to T variant reads.

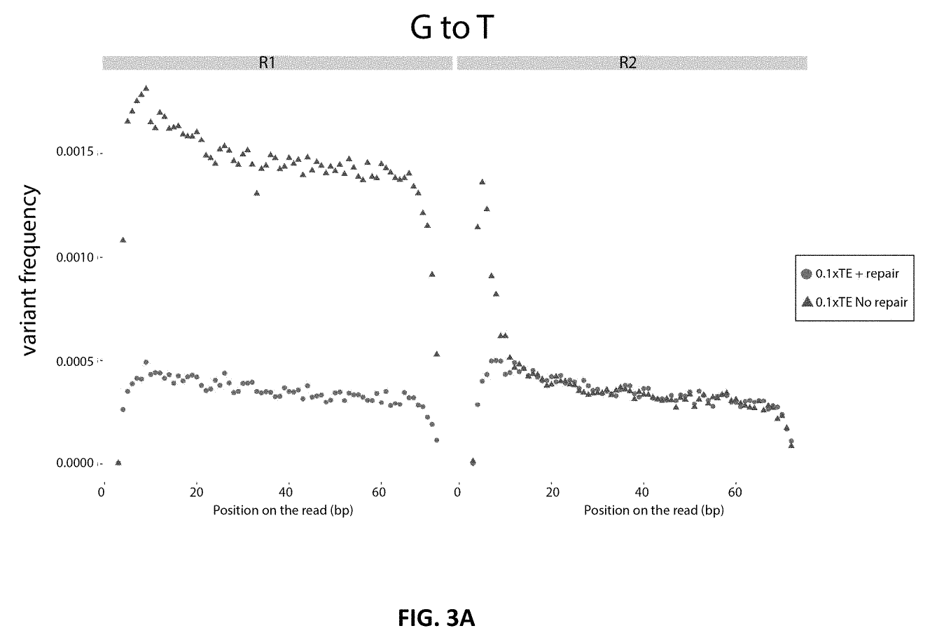

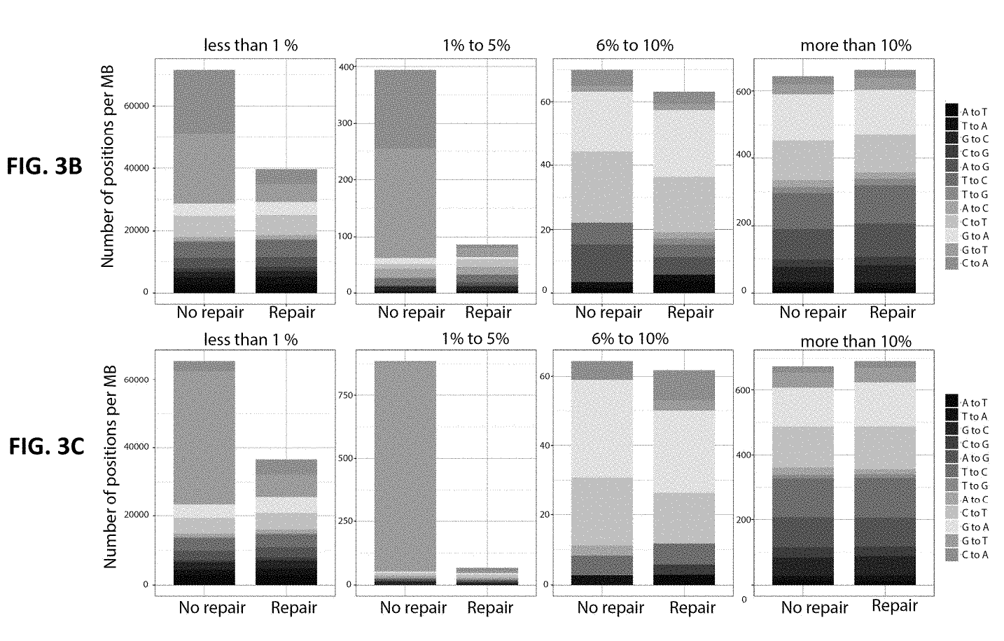

This data showed damage leading to G to T transversions to be stochastic (FIG. 5A). Such stochasticity implies that errors derived from damage are expected to be present at low allelic fractions. Therefore, the identification of low frequency variants, e.g. somatic variants, would be affected by damage while variants present at higher frequency, e.g. germline variants, would be unaffected. To evaluate how damage affects somatic variant identification, we repeated the oxidative damage experiments using common library preparation procedures. We further performed target enrichment using a commercial cancer panel probe set to achieve high sequencing depth of 151 annotated cancer genes (FIG. 3A).

Candidate variants were grouped according to frequency with very low (<1%), low to moderate (1%-5%), medium (6-10%) and high (>10%) frequency classes. We found DNA repair eliminates 77% and 82% of G to T/C to A variant positions in the very low and low to moderate frequency classes, respectively, indicating that those positions were erroneous and due to damage (FIG. 3B). Importantly, most candidate variant positions in the low to moderate frequency class were due to damage despite harboring multiple evidences of variant reads (>=3). The imbalance of G to T compared to C to A positions in the unrepaired dataset (FIG. 3C) confirms the role of damage in erroneous variant calling. In the 0.79 Mb region included in the cancer panel, we found 195 genomic locations with low to moderate G to TIC to A variants with 50 marked as deleterious and 7 annotated as nonsense according to PredictSNP2. For comparison, the repaired dataset only contained 12 genomic locations with low to moderate G to T/C to A variants.

These results indicate that more than 180 positions are false positives and are directly confounding the identification of real somatic variants. This corresponds to approximately one erroneous call per cancer gene. In summary, our data demonstrates a direct link between damage and the ability to accurately call variants at very low and low to moderate frequency, a frequency typically found for somatic variants.

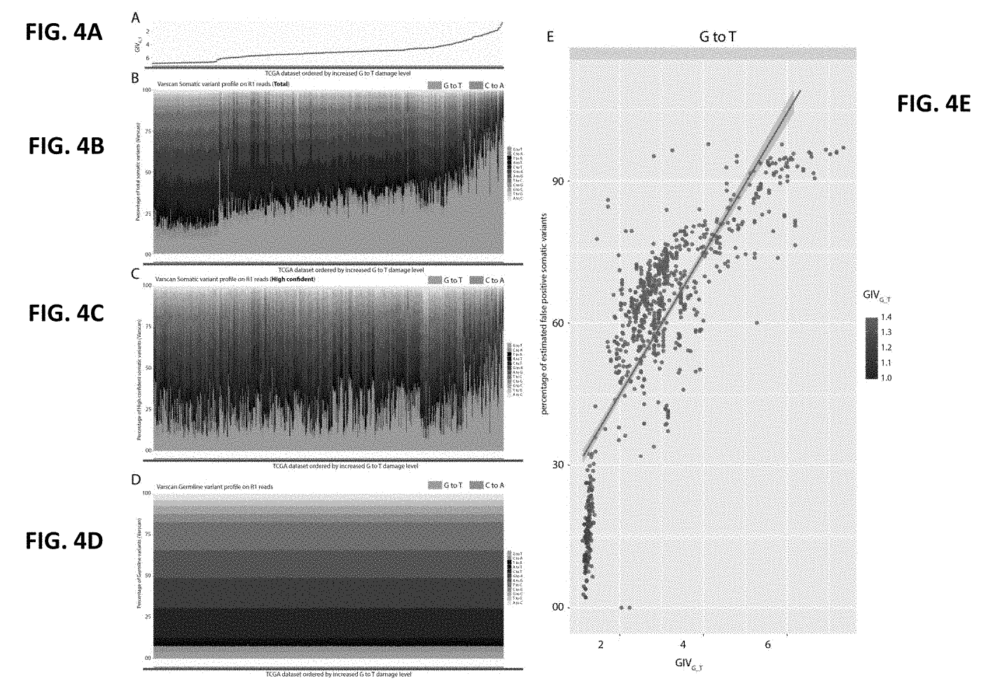

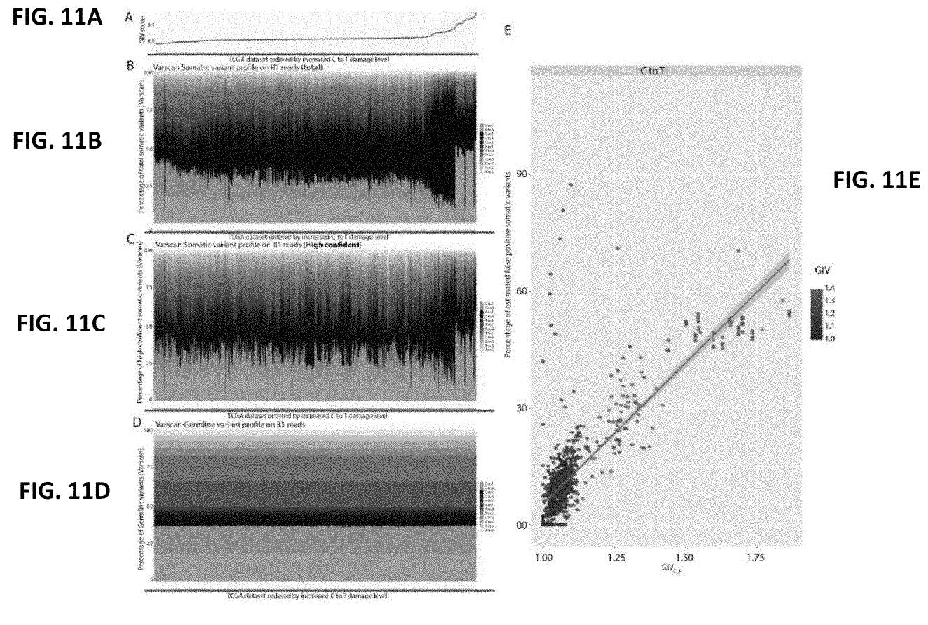

To assess the extent that damage affects somatic variant calls in cancer samples, Varscan, a popular analysis tool, was used to identify germline and somatic variants for all TCGA tumor samples with matched tumor-normal pairs. It was estimated that the effect of damage on both the high confident and total candidate variants identified by Varscan. Prior to variant calling, R1 and R2 reads were independently grouped to assess the global balance of somatic mutation calls between the two groups. Analogous to GIV, an excess of somatic mutation calls in one group represents erroneous calls caused by damage. A large excess of G to T compared to C to A somatic variants was found for most datasets (FIGS. 4A and 4B). Moreover, the fraction of G to T variants compared to other variants increased with the estimated damage measured by the GIV_{G}_{_}_{T} score (FIG. 4B). Importantly, datasets of samples predicted to be severely damaged showed an excess of high confidence G to T somatic variants demonstrating that damage affects high confidence somatic mutation calls in these samples (FIG. 4C). In contrast, the fraction of G to T germline variants was constant across samples and showed no excess in the R1 reads (FIG. 4D), as expected for high frequency variants. Next, we estimated the false positive rate of somatic variant calls and found that 78% of tumor samples have more than 50% false positive G to T somatic variant calls. Furthermore, the percentage of false positives strongly correlated (r=0.79) with the estimated damage in tumor samples (FIG. 4E). This correlation between damage and false positive variant calls indicated that damage is a direct cause of erroneous identification of somatic variants. A smaller subset of the TCGA dataset was also identified with a large excess of both total and high confidence somatic variant calls of the C to T type (FIG. 11). Together, these results highlight a major confounding effect of damage, including high confidence somatic mutation calls in the TCGA datasets.

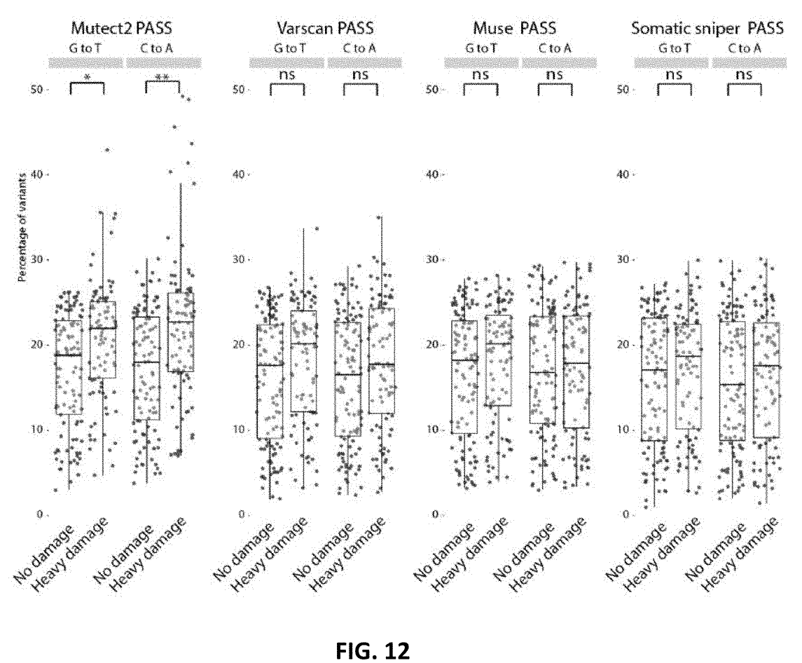

Finally, it was evaluated how damage is affecting current TCGA reference variant files. We downloaded the lung adenocarcinoma vcf files that the TCGA recently generated as part of their annotation workflow and focused on high confident variant calls that passed all filters. Focusing on damage leading to G to T, we classified samples as weak or no damage (GIV_{G}_{_}_{T}<1.5) and heavy damage (GIV_{G}_{_}_{T}>4.5). The heavy damage group showed an overall moderate increase in the fraction of G to T and C to A candidate variants for all callers with Mutect2 (17) showing significant (p<0.05) difference in distributions (FIG. 12). Mutect2 variant profiles displayed a 9% average increase in the fraction of variants being either G to T or C to A in datasets predicted to be heavily damaged compared to weak or no damage datasets suggesting large numbers of variants called with high confidence are derived from artifactual damage. This result is predicted to impact the accurate identification of individual loci and may lead to incorrect diagnostic conclusions in those damaged samples.

To distinguish true from artifactual somatic variants standard strategies include increasing sequencing coverage, setting stringent variant frequency thresholds and applying post-processing computational filters to derive high confident variant calls. These stringent criteria can minimize the effect of damage detected genome-wide as seen for the TCGA variant profiles. But applying stringent criteria does not guarantee to eliminate all errors from damage and more importantly can increase the false negative rate. For example, variant calling algorithms can include strand bias to eliminate artifacts but when faced with limited numbers of variant reads there is a reasonable chance that all evidence reads derived from the same strand orientation, even for genuine variants. Thus, filtering steps are de-facto inferior substitutes to preventing mutagenic DNA damage from occurring in the first place.

In this work, DNA repair has been used to specifically eliminate oxidative damage in our experimental setup for the purpose of evaluating the GIV score and understanding how damages affects variant calling. Additional work will be required to properly identify conditions that will be effective in eliminating damage from TCGA and 1000 Genomes Project samples, and sequencing samples in general.

### Materials and Methods

Materials:

Human liver tumor genomic DNA used in this study was obtained from BioChain Institute, Inc. (Newark, Calif.).

Illumina Library Preparation without and with DNA Repair:

Human liver tumor genomic DNAs were diluted eleven fold using water or various buffers (Table 1 below), and fragmented to 200 bp average size by sonication using a Covaris S2 (Covaris, Woburn, Mass.) ultrasonicator using the following settings: 10% duty cycle, intensity 5, 200 cycles per burst and treatment time of 6 minutes. Without DNA repair, libraries were constructed using either the NEBNext® DNA Library Prep Master Mix Set for Illumina® or the NEBNext Ultra™ II DNA Library Prep Kit for Illumina (New England Biolabs, Ipswich, Mass.). Input DNA was 800 ng for GIV validation library construction and 50 ng for shearing buffer composition DNA libraries. The library quality was assessed using a high sensitivity DNA chip on a Bioanalyzer® (Agilent Technologies, Santa Clara, Calif.). For in vitro DNA repair, prior to the end repair step, the fragmented DNAs were treated with the repair mix at 20° C. for 15 minutes as described in the manual for the NEBNext FFPE DNA Repair Mix (New England Biolabs, Ipswich, Mass.). All other steps in the library preparation protocol were kept the same as for the unrepaired DNA library construction. All libraries were indexed and paired-end sequenced on an Illumina MiSeq® platform.

TABLE 1
DNA dilution buffer compositions

| Sample | Dilution buffer before shearing |
|---|---|
| 1 | 1x TE (10 mM Tris-HCl, pH 8, 1 mM EDTA) |
| 2 | 10 mM Tris-HCl, pH 8, 0.1 mM EDTA |
| 3 | 10 mM Tris-HCl, pH 8 |
| 4 | 0.1x TE (1 mM Tris-HCl, pH 8, 0.1 mM EDTA) |
| 5 | 1 mM Tris-HCl, pH 8 |
| 6 | Water, pH 5.8 |

Deep Sequencing of Cancer Gene Panel from Frozen Human Liver DNA:

A panel of 151 cancer genes (CearSeq® Comprehensive Cancer panel from Agilent Technologies, Santa Clara, Calif.) was captured from human liver tumor genomic DNA using the SureSelect®XT Target Enrichment kit (Agilent Technologies, Santa Clara, Calif.) following the manufacturer's protocol with slight modifications. DNAs were diluted in 0.1×TE buffer and sheared to 200 bp using a Covaris S2 as described earlier, 250 ng of fragmented human liver genomic DNA was subjected to library preparation using the NEBNext Ultra II DNA Library Prep Kit for Illumina protocol without and with DNA repair. Without DNA repair, the fragmented DNA was blunted and dA tailed followed by adaptor ligation. With DNA repair, prior to DNA blunting and dA addition step, the same amount of fragmented DNA was treated with 2 μl of NEBNext FFPE DNA Repair Mix in the NEBNext Ultra II End Prep reaction buffer supplemented with NAD+ at 20° C. for 30 minutes. Then 2 μl of NEBNext Ultra II End Prep Enzyme mix was added to the same reaction to perform blunting and dA tailing followed by adaptor ligation as described in the manual. Pre-captured libraries were amplified with six cycles of PCR using the NEBNext multiplex PCR primers and the NEBNext Ultra II Q5® Master Mix (New England Biolabs, Ipswich, Mass.). Libraries were quantified on an Agilent BioAnalyzer. 750 ng of each pre-captured library (with and without DNA repair) was hybridized to the ClearSeq Comprehensive Cancer XT capture library, as described in the manual of the SureSelect Target Enrichment System for Illumina paired-end sequencing library protocol. Post-captured DNA libraries were amplified with NEBNext multiplex PCR primers and Ultra H Q5 Master Mix for fourteen cycles, Two libraries (with and without DNA repair) were pooled and sequenced on an Illumina MiSeq sequencer at 2×75 bp, Data from a total of four MiSeq runs were combined and analyzed for variant calling.

All Sequencing reads were deposited in ENA under the accession number: PRJEB16681.

Data Analysis:

Paired-end reads were obtained from each sample and adaptors were trimmed using Trimgalore. Reads were paired-end mapped to the hg19 version of the human genome using BWA-MEM with default parameters (k=1). In some cases, local realignments were performed using the GenomeAnalysisTKLite from GATK (IndelRealigner).

After mapping, the map files were organized into two groups, one generated from R1 reads and the other group generated from R2 reads. Mpileup was run independently on both groups with default parameters (and —O -s -q 10 -Q 0).

To correlate the GIV-score with Phred score (FIG. 8A), the same analysis was done as described above with the parameter—qualityscore ranging from 0 to 40 (1 increment).

In the analysis of the cancer panel enrichment experiments, the—max_coverage_limit parameter was set to 5000.

Varscan 2 was run on R1 and R2 using default parameters and—min-coverage 4—min-var-freq 0.08-p-value 0.05—strand-filter 1—min-avg-qual 20.

Using R1 mapping, the false positive rate was estimated using the following equation: 100×((Na−Nb)/(Na+Nb)) with Na being the number of variants of type a and Nb being the number of variants of type b (with b being the complement of a).

### Example 1: The Global Imbalance Value (GIV)

To estimate the number of erroneous variants arising from DNA damage, the directionality of the adaptors used in the Illumina library preparation workflow was employed (FIG. 1A). The P5 flow cell binding site adaptors are ligated to the 5′ end of the original DNA fragment while the P7 adaptors are ligated to the 3′ end of the original DNA fragment. Amplification during PCR or clustering preserves this orientation.

The adaptor directionality permits paired-end sequencing, a process in which both ends of a target sequence are read independently leading to the sequencing of the template strand in the first read (R1) and the reverse complement of the template strand in the second read (R2).

The fact that a damaged base will cause an erroneous base change in only one strand of a DNA duplex was also employed. For example, 8-oxo-dG is a well-known result of oxidative damage often misread by a polymerase as a thymine (T) instead of a guanine (G). Consequently the polymerase incorporates an adenine (A) to pair with 8-oxo-dG. The erroneous variant of 8-oxo-dG is therefore captured as a G to T on R1 reads, while R2 reads capture the reverse complement variant, C to A. Thus, damage leading to a systematic misincorporation of a defined base opposite a damaged base results in a global excess of a variant in R1 when compared to R2. This imbalance is evident when the total number of a variant in R1 is compared to the total number of the same variant type in R2 (FIG. 1A).

These differences were combined and used to calculate the global imbalance value (GIV) score using the following equation:

GIV_{G}_{_}_{T}T=((C1v+C2v)/(C1+C2))/((C1v_RC+C2v_RC)/(C1_RC+C2_RC))

where C1v=Number of G to T variants in R1; C1=Total number of G in R1; C2v=Number of C to A variants in R2; C2=Total number of C in R2; C1v_RC=Number of C to A variants in R1; C1_RC=Total number of C in R1; C2v_RC=Number of G to T variants in R2; C2_RC=Total number of G in R2.

### Example 2: 8-Oxo-dG is Efficiently Repaired by In Vitro DNA Repair

The NEBNext FFPE DNA Repair Mix protocol for in vitro DNA Repair was used. The general strategy for DNA repair by this mix and the related PreCR mix involves two steps: First a DNA glycosylase recognizes a specific damage and cleaves the N-glycosidic bond between the damaged base and the sugar phosphate backbone of the DNA. There are four different glycosylases, each recognizing a particular type of DNA damages. For 8-oxo-dG damage, the glycosylase used is formamidopyrimidine [fapy]-DNA glycosylase (FPG). Cleavage by the specific glycosylase generates an apyrimidinic/apurinic (AP) site in the DNA. Alternatively, AP sites can also arise by spontaneous hydrolysis of the N-glycosidic bond. In either case, the AP site is subsequently processed by E. coli Endonuclease IV, which cleaves the phosphodiester backbone immediately 5′ to the AP site, resulting in a 3′ hydroxyl group and a transient 5′ abasic deoxyribose phosphate (dRP). The second step consists of the incorporation of the correct nucleotides to the 3′ end and removal of the 5′-dRP at the gap accomplished by the action of the DNA polymerase. Finally the nick produced is sealed by Taq DNA ligase, thus restoring the integrity of the DNA.

### Example 3: Shearing Conditions and Oxidative Damage

To introduce variable amount of damage in human genomic DNA we diluted DNA eleven fold with either no buffering (Water, pH=5.81) or different buffering conditions (0.1×TE, 1×TE at pH=8), subjected to acoustic shearing, followed by no repair or repair of the damage by a cocktail of repair enzymes. Libraries from treated and untreated samples were paired-end sequenced using an Illumina MiSeq platform, resulting in an average of ˜4 million paired-end reads per sample. Reads were mapped to a reference human genome (hg19) using BWA-MEM, and mapped sequences were analyzed using our orientation-aware variant calling algorithm followed by GIV scoring. It was found that the frequency of G to T transversions in the same DNA sample varied according to the shearing conditions and was inversely correlated with the buffer concentration used to shear the DNA (FIG. 5A). Treatment of the DNA sample with the repair enzyme cocktail post acoustic shearing reduced the number of G to T variants to baseline levels at all shearing conditions (FIG. 5A and FIG. 6A). Additional excess of G to T variants was observed within the first ˜20 bp of the read R1, consistent with increased oxidative damage on single stranded DNA (FIGS. 1A and 1B). Contrary to a previous study reporting a strong preference for oxidation of G in a CGG context, nucleotide context specificity was observed (FIG. 5B). No effect of EDTA on the levels of DNA damage was found. In addition, several previously uncharacterized signatures of damage that correlated with buffering conditions during shearing were detected (FIG. 5A-5D). Furthermore, the fraction of errors due to damage increased with increasing Phred quality score cutoff (FIG. 7A).

To further identify the causative agent for 8-oxo-dG damage during library preparation, human genomic DNA was subjected to various shearing conditions (table 1, above). Based on the degree of G to T imbalance, it was found that 10 mM Tris pH 8+1 mM EDTA (1×TE) shearing buffer minimized oxidative damage but did not completely abolish it. It was found that shearing in a 10 mM Tris, pH 8 buffer alone was sufficient to bring the G to T imbalance down to almost the same level as 1×TE. The addition of EDTA in the Tris buffer marginally decreased the G to T imbalance (FIG. 6A).

In addition, several previously uncharacterized signatures of damage that correlate with buffering conditions during sonication were detected. The first signature was characterized by an excess of A to T variants located on the first 20 bp of the 5′ end of the R2 sequences. This excess of A to T was reduced in repaired samples. Translated to the original DNA fragment, this damage lead to a T to A transversion at the 3′ end of the fragments. Similar to the G to T signature, this signature was stronger in an unbuffered shearing solution and diminished with additional buffering capacity. Unlike G to T, this signature had strong context specificity with a 5′ T being the preferred context (FIG. 5B). Other minor signatures included C to T and G to T variants located on the first 20 bp of the 5′ end of the R2 reads. Both signatures showed strong context specificity (see for example FIGS. 5A-5E).

### Example 4: Other Damages in the TCGA

An A to T imbalance with context specificity and elevated variant frequency in the first ˜20 bp at the 5′ end of the R2 reads (leading to GIV_{T-A}>1.5) was detected in 0.5% of the TCGA dataset (FIG. 9). Translated to the original DNA fragment, this damage leaded to a T to A transversion at the 3′ end of the fragments and therefore a GIV_{T}_{_}_{A}>1.5. The A to T and G to T signature profiles in public datasets matched those obtained in our validation experiments in which oxidative damage was introduced during library preparation (FIG. 5B). Other imbalances were found, including a C to T imbalance (3% of datasets), consistent with cytosine deamination.

### Example 5: ClearSeq Target Enrichment Analysis

A panel of 151 cancer genes was captured using the ClearSeq Comprehensive Cancer panel and sequenced on an Illumina MiSeq platform using 2×75 paired-end protocol.

Trimming and mapping of the reads were done as described in the Supplementary Materials and Methods section. The reads were locally remapped using GATK IndelRealigner. Reads were classified as R1 or R2 and variants were called using samtools mpileup with the following parameters: —I —O -s -q 10 -Q 30. We used the annotation file corresponding to the ClearSeq Comprehensive Cancer panel provided by Agilent for the —I parameter. For each genomic position, the coverage was downsampled to exactly 300 fold by randomly sampling the mpileup file for all, R1 or R2 reads and the frequency of each variant was calculated.

The overall measurement of damage in the enriched DNA without DNA repair was in agreement with whole genome sequencing (GIV_{G}_{_}_{T capture}=4.4 and GIV_{G}_{_}_{T genome}=3.7). DNA repair fully abolished detectable damage (FIG. 3A).

Next, the coverage at each genomic position was normalized to 300-fold and the variants were classified according to frequency with very low (<1%), low to moderate (1%-5%), medium (6-10%) and high (>10%) frequency variant classes. We found that DNA damage had an effect on very low and low to moderate frequency G to T and C to A variant positions (FIG. 3B). Interestingly, when investigating the total variants profiles, the major effect of damage was reflected in the low to moderate frequency variant class. Indeed, the repaired sample showed 78% less total variants compared to the damaged sample in the low to moderate frequency variant class as opposed to only 44% less total variants in the very low frequency variant class (FIG. 3B). As expected, high frequency variants such as SNP's (>10%) were not affected by damage because the rate of variant calling and variant profiles were similar between the matched repaired and unrepaired samples (FIG. 3B).

What is claimed is:

1. A method for reducing sequencing errors caused by mechanical DNA fragmentation, comprising:
(a) obtaining a DNA sample;
(b) mechanically fragmenting the DNA sample to produce a template;
(c) treating the fragmented DNA with a plurality of DNA repair enzymes; and
(d) obtaining a sequence of the fragmented DNA,
wherein step (c) reduces the number of fragmentation induced errors.

2. The method of claim 1, wherein step (b) is done by acoustic shearing.

3. The method of claim 1, wherein the treating is done by treating the template with enzymes that include at least one DNA glycosylase, an AP endonuclease, a DNA polymerase and a ligase.

4. The method of claim 3, wherein the enzymes are in a mixture.

5. The method of claim 3, wherein the at least one DNA glycosylase comprises oxoguanine glycosylase (OGG) or formamidopyrimidine-DNA glycosylase (FPG).

6. The method of claim 1, wherein step (d) comprises amplifying the template.

7. The method of claim 1, wherein step (d) comprises circularlizing the template.

8. The method of claim 1, further comprising analyzing sequence reads to identify a sequence variation.

9. The method of claim 1, wherein the DNA sample made from a clinical sample.

10. A method for detecting damaged DNA in a sample, comprising:
preparing a sequencing library from the damaged DNA;
sequencing the sequencing library to obtain a plurality of first Watson end sequence reads (R1s) and plurality of second complementary Crick sequence reads (R2s);
for a selected locus, identifying those R1s and R2s that have a sequence abnormality; and
detecting an imbalance between the number of R1s that have a sequence abnormality and the number of R2s that have a complementary sequence abnormality at the same position.

11. The method of claim 10, wherein the damaged DNA is from a clinical sample.

12. The method of claim 11, wherein the clinical sample is a formalin fixed paraffin embedded (FFPE) sample.

13. The method of claim 10, wherein the damaged DNA is fragmented.

14. The method of claim 13, wherein the damaged DNA is fragmented by acoustic shearing.

15. The method of claim 10, wherein the sequence abnormality is a G to T transversion.

16. The method of claim 10, further comprising eliminating sequence reads, or sequence abnormalities, from further analysis if they are associated with an imbalance.
